(19) United States
(12) Patent Application Publication
(10) Pub. No.: US 2009/0232864 A1
(43) Pub. Date: Sep. 17, 2009

(54) METHOD FOR APPLYING A BIOACTIVE, TISSUE-COMPATIBLE LAYER ONTO SHAPED ARTICLES AND THE USE OF SUCH SHAPED ARTICLES

(75) Inventor: Christoph Schultheiss, Pfinztal (DE)

Correspondence Address: DARBY & DARBY P.C., P.O. BOX 770, Church Street Station, New York, NY, 10008-0770, US

(73) Assignee: Forschungszentrum Karlsruhe GmbH, Karlsruhe (DE)

(21) Appl. No.: 12/306,404

(22) PCT Filed: May 15, 2007
(86) PCT No.: PCT/EP07/04295

§ 371 Date: Dec. 23, 2008

(30) Foreign Application Priority Data
Jun. 23, 2006 (DE) 10 2006 028 856.4

(51) Int. Cl. A61K 9/00; C23C 14/34; A61P 43/00; A61B 5/07

(52) U.S. Cl. 424/422; 204/192.11; 600/302

(57) ABSTRACT

A method for applying a bioactive, tissue-compatible layer onto a shaped article includes providing, as a target material, a bioactive glass ceramic having a S53P4 composition, cleaning a shaped article and activating a surface of the shaped article with ions. The Shaped article is exposed to a bioactive glass ceramic beam pulse ablated by a pulsed electron-beam ablation of the bioactive glass ceramic target material so as to deposit a bioactive glass ceramic layer having a thickness ranging from 1 μm to 10 μm on the surface of the shaped article. A respective pulse frequency of the at least one of: 1) an electron pulse of the pulse electron beam abalation, and 2) the bioactive glass ceramic beam pulse, is controlled. Prior to an implantation, so as to avoid an initial cytotoxicity, at least one of the following is performed: a) an exposure of the article at least the times to 24-hour contact with at least one of a culture medium (SBL), a de-mineralized water, and a 0.9% NaCl electrolyte, and b) a wet-vapor sterilization of the article.

### CROSS REFERENCE TO PRIOR APPLICATIONS

This is a U.S. National Phase application under 35 U.S.C. §371 of International Application No. PCT/EP2007/004295, filed on May 15, 2007, and claims the benefit of German Patent Application No. 10 2006 028 856.4, filed on Jun. 23, 2006. The International Application was published in German on Dec. 27, 2007 as WO 2007/147462 A2 under PCT Article 221(2).

### FIELD

The invention relates to a method for at least partially applying a bioactive, tissue-compatible layer onto shaped articles, it also relates to such shaped articles, and to the use of shaped articles coated in this manner.

### BACKGROUND

A class of substances that can be implanted into living tissue and that also have the potential to bond to the soft tissue is referred to as bioactive glass ceramic. Bioactive glass ceramic is an amorphous bioceramic that, in addition to the typical glass components such as sodium, silicon and calcium, also contain small amounts of phosphorus. In particular, this ceramic is especially used in the form of an S53P4 composition containing 53% SiO₂, 4% PO₂, about 21% CaO, about 21% Na₂O as well as other physiological salts. Researchers at the University of Florida have demonstrated that, within narrow ranges of the mixing ratio, not only osteointegration of the implant with the surrounding bone occurs, but the soft tissue can also bond to the surface of the bioactive glass ceramic.

Since bioactive glass ceramics are very brittle and cannot be made into implants or parts thereof, they are employed mainly in particulate form, for purposes of regenerating bone in the jaw area. The implants can be coated by immersing them into liquid bioactive glass ceramic, but this requires high-temperature resistance on the part of the implant material. Other methods for coating implants with bioactive glass ceramics are the sol-gel technique and the plasma-spraying technique—both of which exert a high thermal load on the implants—followed by ion-sputtering and laser vaporization, which only play a subordinate role since these two processes alter the glass property or the stoichiometry.

The following can be said specifically about these methods:

With the plasma-spraying technique, bioactive glass ceramic powder is blown through an electric arc and applied onto the implant at a layer thickness of up to about 100 μm. Because of the “turret formation”, such layer thicknesses are necessary in order to ensure that the implant surface is hermetically sealed and tight with respect to the bioactive glass ceramic surface. Nevertheless, it cannot be ruled out that body fluid will penetrate into or creep under the layer. The result is that entire layer regions peel off of the surface of the implant.

The thermal load during production makes demands on the material characteristics of the implant. Plastics, glass and metals having a low melting point are not a possibility and the same applies to implants that already contain electronic components. Another problematic aspect is the decomposition temperature of bioactive glass ceramics which, depending on the literature source cited, lies between 900° C. and 1400° C. [1652° F. and 2552° F.]; the high temperatures in the electric arc easily lead to the formation of undesired mineralogical phases of calcium phosphate. In comparison to this, the temperatures are considerably lower during the production of layers employing the gel-sol technique, but still reach 400° C. [752° F.], so that most plastics are not an option as implant materials.

Ion-sputtering is characterized by low layer-growth rates of just a few angstroms per minute and deviations from the stoichiometry of up to 20%. The main cause for the low layer growth is the electric charging of the non-conductive, dielectric bioactive glass ceramic target by fast positive ions and the resultant reduced sputtering ion current onto the target. Limitations also apply to lasers, since pulsed UV lasers using the ablation process can fundamentally yield high-quality layers but their investment and operating costs restrict their economic feasibility.

Up until now, it has not been possible to apply a coating made of bioactive glass ceramic onto flexible, subcutaneous and transcutaneous tubes such as catheters, etc., or onto sensitive electronic components that are sheathed with dielectric material and that need to be implanted subcutaneously such as, for instance, heart pacemakers.

The exit site of a transcutaneous implant through the tissue and out of the skin is an open wound that constitutes an ever-present site of inflammation. Moreover, the boundary between the implant and the tissue creates a conduit for bacteria and germs leading into the body, so that it is problematic in terms of the potential for infection. Great progress would be achieved if the implant were to bond to the soft tissue. The same holds true of bonding of subcutaneously implanted shaped articles/housings.

So-called transponders can be cited as examples of sensitive, subcutaneous implants. These are electronic receiving and transmitting units that can be employed as identification systems for animals. For this purpose, such a transponder is installed into a dielectric housing that is coated on the outside with bioactive glass ceramic, and this housing is then hermetically sealed and injected or implanted. Coded excitation of the implanted transponder provides an individual identification system.

The bioactive glass ceramic-coated transponder housing bonds to the tissue. The bonding is important because any migration inside the body could give rise to spontaneous complications that are not acceptable for the user. With animals intended for slaughter as well as laboratory animals, the ability to precisely and reliably locate an implanted transponder is a decisive criterion for its use as an identification system.

Up until now, a pulled-over plastic tube has served to try to prevent the migration of implanted housings/shaped articles containing integrated transponders. The plastic, however, causes a proliferative, inflammatory defense reaction during the course of which fibrogenic cells and later an intercellular substance are formed which, in the most favorable case, causes the housing to be encapsulated by scarring. Particularly in the case of laboratory animals, harm to the health of the population due to an identification system is not tolerable. Mention should be made of catheters as subcutaneous implants such as, for example, renal catheters or else catheters for dialysis of the abdominal cavity, which are acquiring ever-greater significance. The most frequently encountered complications here are inflammation of the kidneys or of the abdominal cavity, which can have life-threatening consequences although, of course, the catheter method is often the last resort in helping a patient with a primary disease.

### SUMMARY

An aspect of the present invention is to provide a method for coating temperature-sensitive implants with bioactive, tissue-compatible substances/coatings that bond to the soft tissue. The applied, exposed layer should be liquid-tight and should lend itself to being applied at a prescribed thickness and roughness. It is a further, alternative aspect of the invention to provide coated shaped articles/housings that can be implanted into living organisms without being rejected.

In an embodiment, the present invention provides for a method for applying a bioactive, tissue-compatible layer onto a shaped article so as to provide a contact surface having a property of promoting the colonization, propagation, and/or proliferation of surrounding soft-tissue somatic cells on the layer. The method includes providing, as a target material, a bioactive glass ceramic having a S53P4 composition, cleaning a shaped article and activating a surface of the shaped article with ions. The shaped article is exposed to a bioactive glass ceramic beam pulse ablated by a pulsed electron-beam ablation of the bioactive glass ceramic target material so as to deposit a bioactive glass ceramic layer having a thickness ranging from 1 μm to 10 μm on the surface of the shaped article so that the bioactive glass ceramic layer is capable of eluting ions from a depth of the layer when subsequently contacted with fluids so as to initiate a morphological change to promote cell colonization. A respective pulse frequency of the at least one of: 1) an electron pulse of the pulse electron beam abalation, and 2) the bioactive glass ceramic beam pulse is controlled so as to control a thermal load resulting from condensation heat during the depositing of the bioactive glass ceramic layer. Prior to an implantation, so as to avoid an initial cytotoxicity, at least one of the following is performed: a) an exposure of the article at least three times to 24-hour contact with at least one of a culture medium (SBL), a de-mineralized water, and a 0.9% NaCl electrolyte, and b) wet-vapor sterilization of the article at at least one of a temperature of at least 134° C. for at least 13 minutes and a temperature of at least 121° C. for a period of at least 30 minutes.

### BRIEF DESCRIPTION OF THE DRAWINGS

The present invention will be described in greater detail on the basis of exemplary embodiments with reference to the FIGURE in which:

FIG. 1: shows a the coating installation.

### DETAILED DESCRIPTION

The method for at least partially applying a bioactive, tissue-compatible layer onto shaped articles in order to create a contact surface having the property of promoting the colonization and propagation as well as the proliferation of surrounding soft-tissue somatic cells on the layer is characterized in that bioactive glass, bioactive glass ceramic in the form of the S53P4composition, is used as the target material. The shaped articles 5 can be cleaned and the surfaces to be coated can be activated with ions. The shaped articles 5 can be exposed on an exposure stage to a bioactive glass ceramic beam pulse 7 ablated by pulsed electron-beam ablation of a bioactive glass ceramic target 3. In this process, the thermal load caused by condensation heat during the bioactive glass ceramic deposition onto the surface of the housing 5 can be controlled via the pulse frequency of the electron pulse 2 or of the bioactive glass ceramic beam pulse 7 until a bioactive glass ceramic layer with a thickness ranging from 1 μm to 10 μm is deposited on the provided housing surfaces, making the layer so thick that, when it subsequently comes into contact with fluids, ions can be eluted out of the depth of the bioactive glass ceramic layer, initiating the morphological change of the surface needed for cell colonization.

With an eye towards avoiding an initial cytotoxicity, prior to the implantation, the bioactive glass ceramic-coated shaped article can either be exposed at least three times to 24-hour contact with a culture medium (SBL), de-mineralized water or 0.9% NaCl electrolyte, or else it can undergo wet-vapor sterilization at a temperature of, for example, at least 134° C. [273.2° F.] for at least 13 minutes, or at a temperature of at least 121° C. [249.8° F.] for a period of at least 30 minutes.

The shaped articles can be dielectric and made of plastic, they can have an ampoule-like or lenticular shape and form a tube or else sheathe a wire and can be of a size that is conducive to implantation into tissue without affecting the natural functions. The shaped articles can be made of glass, ceramic—both dielectric—or of metal—and thus electrically conductive—and they can likewise be of a shape and size that is conducive to implantation into tissue without affecting the natural functions.

The surface of the shaped article/housing to be coated can be activated with Ar, O or N ions or else with ions that are suitable for the process or with their ionized molecules. After at least three instances of contact with fluids, the implant can be sterilized by means of a dry method such as flushing with methylene oxide gas.

The present invention also describes the use of shaped articles/housings coated in this manner. A dielectric or metallic shaped article can be used for injection or transcutaneous/subcutaneous implantation into a living body/organism, where such an article can bond to the adjacent tissue at the site of deposition or at the transcutaneous entry site into the body, without causing a rejection reaction. The shaped article can be employed for diagnosis or therapy after an appropriate medium has first been placed therein. The shaped article/housing can also be used to physically stimulate the body/organism/organs or to transmit measured data or else as a locating medium into which an appropriate electronic therapeutic device or transponder device has first been placed.

An aspect of the method of the present invention is the texture of the surface of the shaped article/housing to be coated. It can be advantageous to activate the surfaces with Ar, O or N ions or their ionized molecules.

The provision of the rough surface so as to attain better adhesion of the tissue cells and bonding to the implantation site can accelerate the solubility of the bioactive glass ceramic. For this reason, the thinnest places of the layer, that is to say, the valleys in the roughness, should be sufficiently thick, for example they should not be less than 1 μm.

The method of the present invention can be executed with a device for generating pulsed, magnetically self-focusing electron beams as described in German patent specification DE 42 08 764 C2. These electron beams can be used in an ablation process to remove material from the dielectric target and to make it bond to an exposed surface. The capability of the electron beam to remove dielectric material is closely related to the property of the magnetically self-focused electron beam to propagate in a dielectric gas. In this process, the dielectric background gas is ionized by the beam and a column of positive gas ions that serves to neutralize the space charge is formed in its propagation channel.

When the beam strikes a bioactive glass ceramic target, the beam electrons ionize the molecules in the area of the surface of the solid object. Thermal electrons from the ionization process escape from depths of 200 nm from the initially very dense ablation plasma to the outside, thus preventing an accumulation of the negative space charge that was entrained by the beam and that would repel the beam.

The energy of the electrons in the beam is between 10 keV and 15 keV, the time duration is about 100 ns and the transported energy is about 1 J. The cross section of the beam is about 10 mm² when it strikes the target surface. This corresponds to a power density of about 10⁸ watt/cm². In accordance with the range of 10 keV to 15 keV electrons, they penetrate the target to a depth of approximately 200 nm (density=3 g/cm³) and deposit the energy in this thin layer. The result is an energy load of the 200 nm-layer corresponding to a specific energy of about 60 kJ/g. In reality, the effective specific energy is less because heat already dissipates into deeper regions of the target during the deposition. Since bioactive glass ceramics have a sublimation energy in the order of magnitude of somewhat more than 10 kJ/g, an explosive removal of the layer, the ablation, takes place. The removed weight per shot is in the range of 10 μg.

The removed bioactive glass ceramic material only partially escapes on the molecular level. Most of it is present in the form of hot, boiling droplets whose size distribution reaches 40 nm. As a result of the pressure conditions during the ablation, the vapor and the droplets normally move away from the target surface. The speed distribution is large; for ions that are double-positively charged, speeds of up to 10 km/s have been measured, followed by single-positively charged ions (1 km/s). Molecules, molecule dusters and droplets follow, whereby the droplets still move at speeds of 100 m/s.

The surface of the implant should be treated prior to the coating. Once the implant has been cleaned and degreased, it can be exposed to the beam of an ion-beam extraction source so as to free the surface of adsorbates and to activate it. The ion energy and current density fall within the normal range and are approximately 3 keV to 5 keV at 10 mA/cm². The ion can be selected as desired, provided that it is non-metallic; however, due to their easy availability, ions of the Ar, O or N type or their ionized molecules are generally used if available. Another familiar method of surface cleaning is a high-frequency treatment or RF treatment (RF=radio frequency).

A substrate can display layer growth if it is arranged in such a way that it is struck by the ablated components. The molten droplets can be homogeneously integrated into the layer because they flow upon impact. The typical growth rate of the layer per shot is 1 nm, and the distance between the target and substrate is typically 6 cm.

Ablation is considered to be among the “cold” coating methods because, in the ideal case, only the surface layer of the target affected by the energy application is ablated and the area underneath it is not heated up. During the coating, the amount of heat input per unit of time onto the shaped article/housing, the implant, the dielectric element, the dielectric elastic element is only determined by the heat content of the precipitated ablation vapors per pulse, multiplied by the pulse repetition frequency. The heating of the substrate can be controlled via the selection of the pulse repetition frequency.

The stoichiometric deviation of the composition of bioactive glass ceramics in the layer is slight. Within the scope of Rutherford backscattering experiments, deviations of typically 5% are measured, so that these are in the vicinity of the resolution limit of the measuring method. The reason for the slight deviation can be ascribed to the fact that the stoichiometry is retained in the droplets.

The stoichiometry of the target and that of the coatings made on this basis constitute parameters. The selected starting substrate of the present invention was therefore a commercially available bioactive glass ceramic bearing the designation S53P4, containing 53% silicon and 4% phosphorus. After unavoidable losses of the readily evaporable sodium fractions during the ablation in the layer, this glass mixture lies within the range of the desired stoichiometry.

The result of the mechanical tests in terms of the adhesion and scratch resistance on dielectric surfaces, on a titanium alloy as well as on PDMS (polymethyl methacrylate or silicon rubber employed in medicine) proved to be positive.

Elution experiments meant to show the leaching of calcium, sodium, silicon and phosphorus ions in response to the morphological change of the coated surface following contact with simulated body fluid yielded positive results. They show that crystallites of hydroxylapatite and several other calcium phosphates formed on the surface of the coating, said crystallites being a prerequisite for the bonding and proliferation of soft tissue cells.

An unexpected finding with bioactive glass ceramics was that the cell adhesion and the cell growth of L 929 mouse fibroblast cells on the layer surface in simulated body fluid (SBF) were disrupted and that the coatings proved to be cytotoxic in standard in-vitro experiments according to ISO 10993-5. The cytotoxicity could be traced back to an elevated silicon concentration due to contact with the simulated body fluid. This increased solubility and ion concentration in the SBF do not occur with the commercially available S53P4 bioactive glass ceramic, but only on the layer. The so-called frozen energy during the production of the layer through ablation—which strongly favors the elution behavior of ions—may be seen as the cause of this.

Another aspect of the present invention is the pre-treatment of the layer. The elution capacity was reduced to normal values, for example, after the surface was brought into contact three times with the culture medium (SBF), de-mineralized water or 0.9% NaCl electrolyte for 24 hours. Furthermore, it has been found that the so-called wet-vapor sterilization, for example, at 134° C. [273.2° F.] for a period of 18 minutes or at 121° C. [249.8° F.] for a period of 30 minutes, has the same effect, which entails the advantage that the most cost-efficient sterilization and the requisite pretreatment can be performed in one work step.

Preliminary quality tests relating to the bio-compatibility of bioactive glass ceramic-coated implants/shaped articles that were produced with the pulsed electron-beam method and implanted in rats yielded successful results. Coated and uncoated substrates were implanted in rats and the tissue reactions were examined after two and four weeks. In each case, implants made of a titanium alloy and of PDMS (silicon rubber) were coated, and uncoated ones were employed as a reference. No macroscopic signs of incompatibility (uncoated, coated) were ascertained for either implantation time period. The histological findings show that, two weeks after the implantation, the titanium implants were slightly encapsulated within a limited and relatively non-inflammatory reaction. There were no signs of de-lamination of the layer. By the same token, the coated as well as uncoated silicon implants exhibited an acceptable, low level of inflammation. When the implants were being prepared, it was observed for the first time that the layer had become de-laminated from the substrate and that it was attached to the soft tissue or had become bonded to it. This is seen as an indication of the desired adhesion between the layer and the soft tissue. Likewise noteworthy is the macroscopic finding that, after four weeks, small blood vessels had formed around the implant site, ensuring that the tissue around the implant was healthy and being supplied with nutrients.

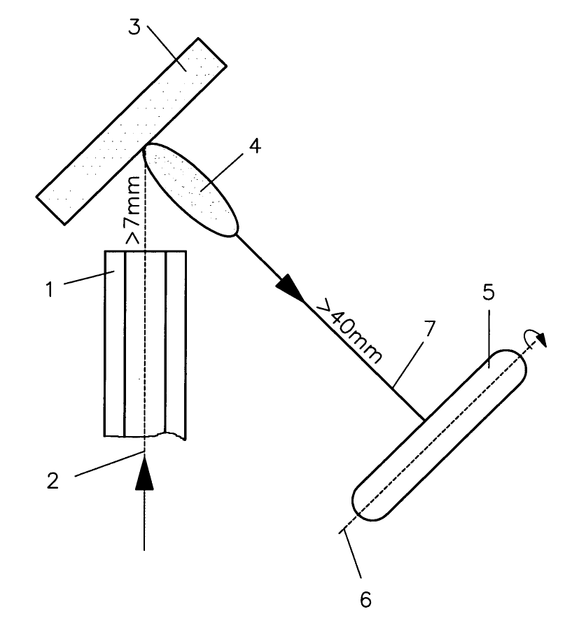

FIG. 1 schematically shows the coating installation. The electron-beam source is only indicated by its outlet, the pipe 1, where the electron beam 2 forms as a channel spark. The electron beam 2 exits from this pipe 1 and strikes the bioactive glass ceramic target 3, where it ablates the coating substance. From the plasma lobe 4 formed in front of the target 3, the ablation components are then hurled in a more or less molecular, cluster-like or droplet-shaped form—depending on the energy feed—in the direction of the implant, shaped article or housing that is to be coated. The distance between the target and the shaped article 5 is at least 40 mm. The implant 5, glass in this example, has an ampoule-like shape and rotates around its longitudinal axis 6 so that a uniform layer thickness can be achieved during the coating procedure.

### LIST OF REFERENCE NUMERALS

pipe

electron beam

bioactive glass ceramic target

plasma lobe

shaped article, housing, implant

longitudinal axis

bioactive glass ceramic beam pulse, longitudinal axis of the ablation beam

1-8. (canceled)

9: A method for applying a bioactive, tissue-compatible layer onto a shaped article so as to provide a contact surface having a property of promoting colonization, propagation, and/or proliferation of surrounding soft-tissue somatic cells on the layer, the method comprising:
providing, as a target material, a bioactive glass ceramic having a S53P4 composition;
cleaning a shaped article;
activating a surface of the shaped article with ions;
exposing the shaped article to a bioactive glass ceramic beam pulse ablated by a pulsed electron-beam ablation of the bioactive glass ceramic target material so as to deposit a bioactive glass ceramic layer having a thickness ranging from 1 μm to 10 μm on the surface of the shaped article so that the bioactive glass ceramic layer is capable of eluting ions from a depth of the layer when subsequently contacted with fluids so as to initiate a morphological change to promote cell colonization;
controlling a respective pulse frequency of at least one of: 1) an electron pulse of the pulse electron beam abalation, and 2) the bioactive glass ceramic beam pulse, so as to control a thermal load resulting from condensation heat during the depositing of the bioactive glass ceramic layer; and
prior to an implantation, so as to avoid an initial cytotoxicity, performing at least one of a) an exposure of the article at least three times to 24-hour contact with at least one of a culture medium (SBL), a de-mineralized water, and a 0.9% NaCl electrolyte, and b) a wet-vapor sterilization of the article at at least one of a temperature of at least 134° C. for at least 13 minutes and a temperature of at least 121° C. for a period of at least 30 minutes.

10: The method recited in claim 9, wherein the surface of the shaped article is a housing surface.

11: The method recited in claim 9, wherein the shaped article is made from a dielectric material.

12: The method recited in claim 9, wherein the shaped article includes at least one of glass, ceramic and metal.

13: The method recited in claim 9, wherein the shaped article has at least one of an ampoule-like shape, a lenticular shape, a tube shape and a wire sheathe shape.

14: The method recited in claim 9, wherein the activating is performed using at least one of Ar ions, O ions, N ions, and ionized molecules thereof.

15: The method recited in claim 9, wherein the exposure is performed on an exposure stage.

16: A bioactive glass ceramic-coated shaped article for implantation prepared by the process of claim 9.

17: The method recited in claim 9, further comprising, after at least three instances of contact with fluids, sterilizing the bioactive glass ceramic-coated shaped article using a dry method.

18: The method recited in claim 17, wherein the dry method includes flushing with methylene oxide gas.

19: A method of injecting or implanting a bioactive glass ceramic-coated shaped article into a living body/organism, the method comprising:
providing the bioactive article by:
providing, as a target material, a bioactive glass ceramic having a S53P4 composition;
cleaning a shaped article;
activating a surface of the shaped article with ions;
exposing the shaped article to a bioactive glass ceramic beam pulse ablated by a pulsed electron-beam ablation of the bioactive glass ceramic target material so as to deposit a bioactive glass ceramic layer having a thickness ranging from 1 Mm to 10 μm on the surface of the shaped article so that the bioactive glass ceramic layer is capable of eluting ions from a depth of the layer when subsequently contacted with fluids so as to initiate a morphological change to promote cell colonization;
controlling a respective pulse frequency of at least one of: 1) an electron pulse of the pulse electron beam abalation, and 2) the bioactive glass ceramic beam pulse, so as to control a thermal load resulting from condensation heat during the depositing of the bioactive glass ceramic layer; and
performing so as to avoid an initial cytotoxicity, at least one of a) an exposure of the article at least three times to 24-hour contact with at least one of a culture medium (SBL), a de-mineralized water, and a 0.9% NaCl electrolyte, and b) wet-vapor sterilization of the article at at least one of a temperature of at least 134° C. for at least 13 minutes and a temperature of at least 121° C. for a period of at least 30 minutes;
providing the living body/organism; and
at least one of injecting, transcutaneously implanting and subcutaneously implanting the article into the living body/organism so that the article bonds to adjacent tissue at at least one of a site of deposition of the article and a transcutaneous entry site without causing a rejection reaction.

20: The method recited in claim 19, further comprising:
providing at least one of a diagnosis and therapy medium in the article; and
performing a diagnosis or therapy using the article.

21: The method as recited in claim 20 wherein the diagnosis or therapy includes physically stimulating the body/organism using the article.

22: The method as recited in claim 20 wherein the medium includes an electric transponder device, and further comprising transmitting measuring data.

23: The method as recited in claim 20 wherein the medium includes an electronic transponder device configured to transmit a location of the article.
